(19) United States
(12) Patent Application Publication
(10) Pub. No.: US 2003/0149414 A1
(43) Pub. Date: Aug. 7, 2003

(54) Nonwoven web including a superabsorbent region and articles including the same

(76) Inventor: Fouad D. Mehawej, Forest Lake, MN (US)

Correspondence Address: Julie Post, H.B. Fuller Company, 1200 Willow Lake Blvd., P.O. Box 64683, St. Paul, MN, 55164-0683, US

(21) Appl. No.: 10/274,335

(22) Filed: Oct. 18, 2002

Related U.S. Application Data
(63) Continuation-in-part of application No. 10/066,935, filed on Feb. 4, 2002.

(51) Int. Cl. A61F 13/15

(52) U.S. Cl. 604/368; 604/370

(57) ABSTRACT

An absorbent article that includes a nonwoven web that includes synthetic polymer fibers, a first surface, a second surface opposite the first surface, a first region including superabsorbent polymer, the first region extending into the nonwoven web from the first surface toward the second surface, and a second region essentially free of superabsorbent polymer, the second region extending into said nonwoven web from the second surface toward the first surface.

### REFERENCE TO RELATED APPLICATIONS

This application is a continuation-in-part of U.S. patent application Ser. No. 10/066,935, filed Feb. 4, 2002.

### BACKGROUND

The invention relates a nonwoven web having a superabsorbent region.

Absorbent articles such as disposable diapers and feminine hygiene products often include multiple layers including a top sheet, an acquisition layer, a core of compressed cellulose fibers, often referred to as “fluff” or “pulp,” held together with chemical binder, or through physical entanglement and compression, and a back sheet. These individual components are combined during manufacturing to produce the finished article.

The top sheet and acquisition layer are individual components, function to disperse liquid, which enables the liquid to transfer to a greater area of the core, and assist in maintaining a dry feel to the wearer's skin. The top sheet and acquisition layer are discreet components of the absorbent article, often are composed of materials that differ from the material of the absorbent core, and tend to have little to no absorbent capacity.

The absorbent core is designed to absorb and hold liquid. Many efforts have been made to increase the absorbent capacity and rate of absorption of cellulose fiber cores. Superabsorbent polymers in particulate and powder form have been added to disposable diaper and feminine napkin cores to improve the absorbent capacity and rate of absorption of the articles. In the case of a diaper construction, for example, superabsorbent powder or particulate is sifted in with the absorbent core material during the diaper manufacturing process. Many problems are associated with superabsorbent powders. Superabsorbent particles are very fine and tend to become airborne during processing. Superabsorbent particles also generally do not adhere to the substrate and tend to migrate and shift during manufacturing, shipping, handling, use or a combination thereof. The movement of the superabsorbent particles can lead to insufficient liquid storage capacity in some areas and excess liquid storage capacity in other areas of the article.

Cellulose fiber cores have disadvantages in that they have weak integrity in both, dry and wet, conditions. Additional compression and embossing processes designed to improve the integrity of cellulose fiber cores often result in a stiffer core having a poor absorption rate. In addition, during the manufacture of cellulose fiber cores loose fibers become air-borne and may present a safety hazard.

Airlaid or pre-made absorbent cores provide a thinner core product and reduce at least some of the problems related to the processing of loose cellulose fibers, but they tend to lack integrity. Chemical binders are often used to improve the integrity of airlaid cores. However, chemical binders tend to negatively impact the absorption rate and absorption capacity of the core.

### SUMMARY

In one aspect the invention features an absorbent article that includes a nonwoven web that includes synthetic polymer fibers, a first surface, a second surface opposite the first surface, a first region that includes superabsorbent polymer, the first region extending into the nonwoven web from the first surface toward the second surface, and a second region essentially free of superabsorbent polymer, the second region extending into the nonwoven web from the second surface toward the first surface.

In one embodiment, the second region extends into the thickness of the nonwoven web a distance sufficient to prevent the wearer of the article from contacting the superabsorbent polymer of the first region after the superabsorbent polymer has been contacted with liquid.

In some embodiments, the first region is impregnated with the superabsorbent polymer.

In other embodiments, the second region exhibits a property that includes fluid acquisition, fluid dispersion or a combination thereof. In some embodiments, the second region is available for contact with the wearer of the absorbent article.

In another embodiment, the first region is capable of storing fluid.

In some embodiments, the second region includes a first plurality of fibers of a first polymer and a second plurality of fibers of a second polymer.

In other embodiments, the second region is soft to the touch.

In another embodiment, the absorbent article includes a top sheet, and a back sheet, the nonwoven web being disposed between the top sheet and the back sheet. In one embodiment, the back sheet is positioned away from a wearer's skin.

In some embodiments, the back sheet is liquid impervious.

In other embodiments, the back sheet is moisture vapor permeable.

In another embodiment, the first region is substantially coextensive with the second region.

In one embodiment, the article is free of a fluid acquisition sheet or a fluid dispersion sheet.

In other embodiments, a disposable diaper, feminine hygiene product, bandage, wound care product, surgical pad, adult incontinence pad, breast pad, bib or mattress pad that includes an absorbent article described herein.

In one embodiment, the absorbent article that includes an absorbent core that includes a nonwoven web of synthetic polymer fibers, a first surface, a second surface opposite the first surface, a first region that includes superabsorbent polymer, the superabsorbent polymer having been formed in situ, the first region extending into the nonwoven web from the first surface toward the second surface, and a second region essentially free of superabsorbent polymer, the second region extending into the nonwoven web from the second surface toward the first surface.

In another aspect, the invention features an absorbent core dimensioned for use in an absorbent article, the core includes a nonwoven web that includes synthetic polymer fibers, a liquid storage region that includes a superabsorbent polymer formed in situ in the region, the liquid storage region extending into the nonwoven web from a first surface of the nonwoven web toward a second surface of the nonwoven web, and a liquid acquisition region free of superabsorbent polymer, the liquid acquisition region extending into the nonwoven web from a first surface of the nonwoven web toward the liquid storage region.

In another aspect, the invention features an article that includes a nonwoven web that includes a first surface, a second surface opposite the first surface, a fluid storage region includes superabsorbent polymer, the fluid storage region extending into the nonwoven web from the second surface of the web toward the first surface of the web the superabsorbent polymer having been formed in situ in the fluid storage region, and a fluid acquisition region essentially free of superabsorbent polymer, the fluid acquisition region extending into the nonwoven web from the first surface toward the second surface.

In other aspects, the invention features a method of making an absorbent article, the method including impregnating a first region of a nonwoven web with an aqueous composition that includes a superabsorbent polymer precursor and a crosslinking agent, the first region extending from a first surface of the web into the depth of the web such that the second surface of the web opposite said first surface of said web is free of superabsorbent polymer precursor, and drying the composition. In one embodiment, the drying includes exposing the composition to microwave radiation. In another embodiment, the drying includes exposing the composition to heat.

In one embodiment, the method of making an absorbent article includes impregnating a high loft nonwoven web with an aqueous composition that includes a superabsorbent polymer precursor and a crosslinking agent, and drying the composition, the drying including exposing the composition to microwave radiation, wherein the dried absorbent article includes from 10% by weight to about 90% by weight superabsorbent polymer.

The invention features a nonwoven web that can be constructed to provide a fluid acquisition function, a fluid distribution function, and a fluid storage function. The nonwoven web enables the construction of an absorbent article (e.g., personal hygiene article) in which a single component provides a fluid acquisition function, a fluid distribution function, and a fluid storage function.

Other features of the invention will be apparent from the following description of the preferred embodiments thereof, and from the claims.

### GLOSSARY

In reference to the invention, these terms have the meanings set forth below:

“Superabsorbent polymer precursor” means a polymer that is capable of functioning as a superabsorbent polymer upon crosslinking. “Superabsorbent polymer” means a polymer that is capable of absorbing water in an amount that is many times the polymer's weight.

### DETAILED DESCRIPTION

The nonwoven web includes synthetic polymer fibers, a first region that includes superabsorbent polymer and extends from a first surface of the web into the depth (i.e., thickness) of the web, and a second region that is free of superabsorbent polymer and extends from a second surface of the web into the depth of the web, the second surface being opposite the first surface of the web. The first and second regions are preferably continuous across the length and width of the web and extend into the depth of the web a distance less than 100% of the depth of the web. The first and second regions are also preferably contiguous or substantially contiguous with each other.

The superabsorbent polymer containing first region is herein referred to as the superabsorbent region. The superabsorbent region is preferably impregnated with superabsorbent polymer, i.e., superabsorbent polymer is present throughout the three-dimensional matrix of the web in the superabsorbent region polymer can reside on the fibers of the web in the region, in the interstices of the web in the region or a combination thereof.

The depth to which the superabsorbent region extends into the web is referred to herein as the thickness of the superabsorbent region. Preferably the superabsorbent region extends into the web a depth sufficient to provide the desired absorbent capacity and absorption rate for the intended use of the absorbent article. The superabsorbent region of the web preferably includes from about 10% by weight to about 70% weight superabsorbent polymer, more preferably from about 10% by weight to about 70% by weight superabsorbent polymer, most preferably from about 30% by weight to about 60% by weight superabsorbent polymer.

The superabsorbent region of the web exhibits good saline absorption under load, high saline absorption capacity and high water absorption capacity. Preferably the superabsorbent region exhibits a saline absorption capacity under load of at least 10 g 0.9% saline solution/g superabsorbent region under a 0.3 pound per square inch (psi) load, more preferably at least 15 g 0.9% saline solution/g superabsorbent region, most preferably at least 20 g 0.9% saline solution/g superabsorbent region. The superabsorbent region also preferably exhibits a water absorption capacity of at least 20 g water/g superabsorbent region, more preferably at least 40 g water/g superabsorbent region, most preferably at least 70 g water/g superabsorbent region within a period of 10 minutes.

The second region, which is free of superabsorbent polymer, can be of a thickness sufficient to prevent the gel that is formed when the superabsorbent polymer is contacted with liquid from contacting the wearer. The region that is free of superabsorbent polymer can extend into the web to a depth sufficient to provide a soft feel to the touch. The thickness of the region of the web that is free of superabsorbent polymer can be sufficient to provide the function of fluid acquisition, fluid distribution or a combination thereof.

The nonwoven web is preferably a high loft nonwoven web, i.e., a nonwoven web having a density of no greater than 0.01 gram per cubic centimeter (g/cm³). The three dimensional structure of a high loft nonwoven web matrix can include passageways, e.g., channels, through which liquid (e.g., water, blood, and urine) can migrate, e.g., wick. When liquid contacts the superabsorbent polymer, the superabsorbent polymer begins to expand. In some embodiments, the high loft nonwoven web and the fibers of the high loft nonwoven web expand when contacted with liquid. The three-dimensional nature of the high loft matrix and the expansion of the web can accommodate liquid present in the web, liquid traveling into the web, and the swelling superabsorbent polymer. The expansion of the web enables the nonwoven web to absorb a greater volume of liquid relative to a nonwoven web having a relatively high density, and being essentially two-dimensional.

The three-dimensional matrix of the high loft nonwoven web also assists in maintaining the swollen, i.e., gelled, superabsorbent polymer in the web matrix. Preferably the superabsorbent polymer gel does not migrate out of the high loft matrix and does not transfer or move during use of the absorbent article.

The nonwoven web exhibits good dry strength and maintains strength and integrity when wet. Preferably the nonwoven web exhibits a dry tensile strength of at least 2000 g/25.4 mm, more preferably at least 2500 g/25.4 mm, most preferably a dry tensile strength of at least 3000 g/25.4 mm, and a wet tensile strength of at least 150 g/25.4 mm, more preferably, at least 400 g/25.4 mm, most preferably at least 500 g/25.4 mm.

The nonwoven web is preferably a high loft nonwoven web having a basis weight of greater than 22 g/m² for a web thickness (i.e., caliper) of at least 1 millimeter (mm), preferably at least 30 g/m², more preferably at least 60 g/m², more preferably at least 80 g/m², most preferably at least 100 g/m². The high loft nonwoven web can vary in thickness depending on the application. Suitable high loft nonwoven webs have a thickness of at least 10 mm, more preferably at least 15 mm. The high loft nonwoven web also has a density no greater than 0.01 g/cm³, preferably from about 0.002 g/cm³ to about 0.009 g/cm³, more preferably from about 0.007 g/cm³ to about 0.009 g/cm³. Other useful nonwoven webs with loft have a density of no greater than 0.025 g/cm³, and no greater than 0.023 g/cm³.

The nonwoven web includes synthetic polymer fibers of, e.g., polyester, polyolefin (e.g., polypropylene, polyethylene, and copolymers of polyolefins and polyesters), polyamide, polyurethane, polyacrylonitrile, and combinations thereof including copolymers thereof, bicomponent (e.g., sheath core) fibers and combinations thereof. The nonwoven web can also include a region that includes fibers of a different polymer, polymers or combinations of polymers relative to the fiber composition of another region. The superabsorbent region of the web can include superabsorbent polymer fibers. Preferably the nonwoven web is resilient and includes resilient fibers (e.g., polyester fibers). The fibers can be curly and mechanically and physically entangled.

Nonwoven webs can be formed using a variety of methods including, e.g., air-laying, wet laying, garneting and carding, melt blown, melt spinning, and spunbond techniques, and combinations thereof. A variety of bonding systems can be used to bond fibers of a nonwoven web including, e.g., thermal bonding, resin bonding, mechanical bonding (e.g., needle punch and hydroentangling), and combinations thereof.

The superabsorbent polymer present in the first region of the nonwoven web is capable of absorbing water in an amount many times its own weight. Preferably the superabsorbent polymer is capable of absorbing water in an amount of at least 100 times its own weight, and can be capable of absorbing at least 150 times its own weight, more preferably at least 200 times its own weight. The superabsorbent polymer present in the superabsorbent region can be formed in situ, i.e., in place on the nonwoven web, e.g., from an aqueous superabsorbent polymer composition.

The aqueous superabsorbent polymer composition includes a superabsorbent polymer precursor (e.g., an alkali soluble polyelectrolyte) and a crosslinking agent. As the aqueous superabsorbent polymer composition dries, the superabsorbent polymer precursor crosslinks to form the superabsorbent polymer. Particularly useful superabsorbent polymer precursors include polymers of water soluble monomers. Preferably the polymer is an at least partially neutralized polymer derived from α, β-ethylenically unsaturated mono- or dicarboxylic acid monomers, acid anhydride monomners or a combination thereof, and a crosslinking agent. Useful water soluble α,β-ethylenically unsaturated mono- or dicarboxylic acid monomers include, e.g., acrylic acid, methacrylic acid, crotonic acid, maleic acid, maleic anhydride, itaconic acid, fumaric acid and combinations thereof.

The phrase “partially neutralized” refers to the presence of at least some neutralized carboxylic acid groups in the superabsorbent polymer precursor. The superabsorbent polymer precursor can be fully neutralized. The phrase “fully neutralized” means that the superabsorbent polymer precursor composition exhibits a pH greater than 7. The superabsorbent polymer precursor composition is adjusted to a pH from about 5 to about 6 using an alkali metal hydroxide examples of which include sodium hydroxide, calcium hydroxide, potassium hydroxide and combinations thereof. A metal alkoxide can be used in place of the metal hydroxide examples of which include calcium hydroxide and potassium hydroxide. It is preferred to first neutralize from about 50% to about 95% of the acid groups, more preferably from about 65% to about 85% of the acid groups, most preferably from about 75% to 85% of the acid groups with an alkali metal hydroxide to a pH of from 2 to 6, more preferably a pH from 5 to 6, and then further neutralize the superabsorbent polymer precursor with a volatile fugitive alkaline component (i.e., a base) to a pH greater than 7, more preferably from greater than 7 to 10, most preferably from greater than 7 to 8, to achieve a fully neutralized, i.e., 100% of the acid groups are neutralized, superabsorbent polymer precursor.

Suitable fugitive alkaline components include, e.g., ammonia, triethylamine, dimethylethylamine, and combinations thereof. The volatile fugitive alkaline component dissipates as the aqueous composition dries. The dissipation of the base liberates a sufficient portion of the carboxylate groups to the free acid, i.e., carboxylic, form. The carboxylic groups are then available for reaction with the crosslinking agent.

The viscosity of the aqueous superabsorbent polymer composition is preferably from about 50 centipoise (cps) to about 50,000 cps, preferably from about 100 cps, to about 30,000 cps, preferably from about 100 cps to about 20,000 cps, preferably from about 100 cps to about 10,000 cps, preferably from about 100 cps to about 5,000 cps, preferably from about 100 cps to about 2,500 cps at a solids content of about 23%. Preferred aqueous superabsorbent polymer compositions have a viscosity suitable for application using spray techniques, saturation techniques, and coating techniques.

The aqueous superabsorbent polymer composition preferably includes from about 5% by weight to about 65% by weight, preferably from about 10% by weight to about 50% by weight, more preferably from about 20% by weight to about 40% by weight solids.

Useful aqueous superabsorbent polymer compositions are described in, e.g., PCT Patent Application No. WO 00/61642 (Anderson et al.) and U.S. Pat. No. 5,693,707 (Cheng et al.) and incorporated herein. A useful commercially available aqueous superabsorbent polymer composition is available under the trade designation FULATEX PD-8081-H from H.B. Fuller Company (Vadnais Heights, Minn.).

A sufficient amount of crosslinking agent is added to the aqueous superabsorbent polymer composition to achieve the superabsorbent polymer. Preferably the crosslinking agent reacts with the functional groups on the superabsorbent polymer precursor in less than 24 hours during drying at ambient (i.e., about 20° C.) temperature or a higher temperature. Useful crosslinking agents include any substance that will react with the hydrophilic groups of the aqueous solution polymer. Useful crosslinking agents include, e.g., zirconium ions, ferric aluminum ions, chromic ions, titanium ions and combinations thereof, and aziridine. A variety of suitable crosslinking agents are described in U.S. Pat. No. 4,090,013 and incorporated herein. One example of a useful commercially available crosslinking agent is BACOTE 20 ammonium zirconyl carbonate available from Magnesium Elektron Inc. (Flemington, N.J.). Preferably the composition includes crosslinking agent in an amount of from about 2 parts to about 24 parts, more preferably from about 4 parts to about 14 parts, most preferably from about 6 parts to about 10 parts based on the weight of the dry ingredients.

The composition can also include chain transfer agents to alter the molecular weight of the superabsorbent polymer. Suitable chain transfer agents include, e.g., sodium hypophosphite, thioglycolic acid, mercaptans including, e.g., primary octyl mercaptan, 2-mercaptoethanol, n-dodecylmercaptan, n-octylmercaptan, t-dodecyl mercaptan, mercaptoethanol, iso-octyl thioglycolurate, mercapto carboxylic acids having 2 to 8 carbon atoms, and their esters, examples of which include 3-mercapto propionic acid and 2-mercapto propionic acid, halogenated hydrocarbons including, e.g., carbonbromo compounds (e.g., carbon tetrabromide and bromotrichloromethane) and combinations thereof. The chain transfer agent can be present in the aqueous superabsorbent polymer composition in an amount no greater than 5% by weight, preferably from 1% by weight to about 4% by weight, more preferably from 1% by weight to about 3% by weight.

The composition can also include other components including, e.g., wetting agents, defoamers, plasticizers, binder agents, and combinations thereof.

The superabsorbent polymer is applied to the high loft web in the form of an aqueous superabsorbent polymer composition that, upon drying, crosslinks to form the superabsorbent polymer.

The aqueous superabsorbent polymer composition can be applied to the web using a variety of techniques including, e.g., soaking, spraying, printing, coating, and combinations thereof. Preferably the superabsorbent region of the web is impregnated with superabsorbent polymer, i.e., the superabsorbent polymer exists throughout the web matrix.

The aqueous superabsorbent polymer composition can be dried according to various methods including, e.g., air (including, e.g., circulating air), heat, microwave radiation (i.e., radiation in the frequency range of 300 MHz to 300,000 MHz including, e.g., 915 MHz, 2450 MHz, 5800 MHz, and 22,155 MHz,), radio frequency radiation, ultraviolet light radiation, electron beam radiation, and combinations thereof (e.g., by passing the nonwoven web through a circulating air oven).

The superabsorbent nonwoven web is useful in absorbent articles including, e.g., disposable absorbent articles, i.e., articles that are designed to be disposed of after use. Useful applications for the superabsorbent nonwoven web include, e.g., as the core or a component of the core of various absorbent articles including, e.g., disposable diapers, feminine hygiene products (e.g., sanitary napkins and panty liners), bandages, wound care products, surgical pads, incontinence articles (e.g., adult incontinence pads, and incontinence undergarments), garments (e.g., training pants, pull-on garments), breast pads, bibs, perspiration pads (e.g., underarm, wrist and head), collar inserts, shoe inserts, and hat inserts (e.g., hat band). The superabsorbent nonwoven can be used in combination with other core materials including, e.g., cellulose fibers and other fluff materials. The superabsorbent nonwoven can be present in an absorbent article in the form of a continuous web, positioned in discreet regions on another component of an absorbent article, and combinations thereof. The superabsorbent nonwoven can be located in various positions and configurations on or in an article including e.g., randomly or in a pattern (e.g., strips), and combinations thereof. In one embodiment, the superabsorbent nonwoven is positioned such that the superabsorbent region is in contact with cellulose fibers. In other embodiments, the article includes multiple superabsorbent nonwoven webs positioned in alternating relation with regions of cellulose fibers. The superabsorbent nonwoven can also be maintained in position in or on an article through an adhesive composition.

The article in which the superabsorbent nonwoven web is incorporated can optionally include other components including, e.g., a body fluid pervious top sheet, an acquisition layer, a second absorbent layer (e.g., a second core or fibrous layer), a body fluid impermeable back sheet, and combinations thereof.

The top sheet can be compliant, soft feeling, and non-irritating to the wearer's skin. The top sheet can be liquid pervious permitting liquids (e.g., urine) to penetrate through its thickness. A suitable top sheet may be manufactured from a wide range of materials including, e.g. woven and nonwoven webs, films, porous foams, porous films including, e.g., perforated film (e.g., film of polyethylene, polypropylene or a combination thereof). The top sheet can be hydrophobic to isolate the wearer's skin from fluids that have passed through the top sheet and are contained in the absorbent core.

The acquisition layer preferably is capable of dispersing liquid to the surface of the core.

The second absorbent layer may include loose fibers, fibers held together through a binder, compressed fibers and combinations thereof. The fibers of the second absorbent layer can be of a variety of compositions including, e.g., natural fibers (e.g., wood pulp, jute, cotton, silk and wool and combinations thereof), synthetic fibers including (e.g., nylon, rayon polyester, acrylics, polypropylenes, polyethylene, polyvinyl chloride, polyurethane, and combinations thereof), and combinations thereof.

The back sheet can be positioned away from a wearer's skin and prevents the liquids contained in the absorbent core from wetting articles that contact the absorbent article. The back sheet can be impervious to liquid and permeable to moisture vapor.

The superabsorbent nonwoven web can be disposed between any two components of an absorbent article including, e.g., between the body fluid pervious top sheet and a body fluid impermeable back sheet, between an acquisition layer and a body fluid impermeable back sheet, and combinations thereof. The superabsorbent regions of the nonwoven web can be in contact with cellulose fibers. The superabsorbent nonwoven web can also be located in contact with a wearer.

The invention will now be described further by way of the following examples. All parts, ratios, percents and amounts stated in the Examples are by weight unless otherwise specified.

### EXAMPLES

Test Procedures

Test procedures used in the examples include the following.

Total Water Absorbency

The total water absorbency (g/g) is the weight of tap water in grams (g) that each gram of a 100 cm² sample of composite absorbs in 10 minutes. A 100 cm² (10 cm×10 cm) sample of dry composite is weighed (WD). The sample is then submerged in tap water for 10 minutes. The wet and swollen composite is placed on a pre-weighted metal screen (WS) for one minute. The excess water present in the sample is allowed to drain. The wet sample and the screen are then weighed (WW).

The total water absorbency (Twa) is calculated according to the following equation:

Twa=[(WW−WS)−WD]/WD

and reported in g absorbed water/g composite

Total 0.9% Saline Solution Absorbency Under Load

The total 0.9% saline absorbency (g/g) is the weight of 0.9% saline (g) that each gram of a 100 cm² sample of composite absorbs in 10 minutes. The total 0.9% saline absorbency is determined by weighing a 100 cm² (10 cm×10 cm) sample of dry composite (WD). The sample is placed in a receptacle and a metal mesh screen and brass weights are placed on top of the sample. Both the metal screen and the weights have the same size as (i.e., are coextensive with) the sample, and the total weight of the metal mesh screen and brass weights must exert 0.3 psi on the sample. A sufficient amount of 0.9% saline solution is poured into the receptacle to submerge the absorbent sample. After 10 minutes, the weight and metal screen are removed. The absorbent sample (WW) is then promptly weighed.

The total 0.9% saline absorbency under load (AUL) is calculated according to the following equation:

0.9% Saline AUL=(WW−WD)/WD

and reported in g absorbed 0.9% saline solution/g composite

Dry Tensile Strength

A 4 inch×1 inch strip of sample composite is cut and ½inch strips of masking tape are wrapped at each of the 1 inch wide ends of the composite strip. The composite strip is then placed between the jaws of an Instron tester (Instron Corp., Canton, Mass.) and tensile strength is measured at a 12 inch/min cross-head speed. The average tensile strength of 5 samples is reported as the Dry Tensile Strength in g/in.

Wet Tensile Strength

A 4 inch×1 inch strip of sample composite is cut and ½inch strips of masking tape are wrapped at each of the 1 inch wide ends of the composite strip. The composite strip is then soaked in water for 5 minutes, gently patted dry of excess water and then promptly tested by placing the sample between the jaws of an Instron tester. Tensile strength is measured at a 12 inch/min cross-head speed. The average tensile strength of 5 samples is reported as the Wet Tensile Strength in g/in.

% Superabsorbent Polymer (SAP) Loading

The percent superabsorbent polymer present in the composite is determined by weighing the web prior to treatment with superabsorbent polymer, weighing the dried composite after treatment with superabsorbent polymer, subtracting to find the weight of superabsorbent polymer in the composite, and dividing the weight of the superabsorbent polymer by the total weight of the composite.

The results are reported as % SAP.

Controls 1 and 2

Samples were prepared by saturating polyester fiber nonwoven webs having the properties set forth in Tables 1 and 2 with an aqueous superabsorbent polymer composition of 95 parts FULATEX PD-8081-H aqueous superabsorbent polymer (23% solids) (H.B. Fuller Company, Vadnais Heights, Minn.) and 5 parts BACOTE 20 ammonium zirconyl carbonate (40% active as supplied) (Magnesium Elektron Inc., Flemington, N.J.). The webs were dried and weighed to determine % superabsorbent polymer present in the composite.

### Examples 1-4

Superabsorbent composites were prepared by saturating polyester fiber nonwoven webs having the properties set forth in Table 1 with an aqueous superabsorbent polymer composition of 95 parts FULATEX PD-8081-H aqueous superabsorbent polymer (23% solids) and 5 parts BACOTE 20 ammonium zirconyl carbonate (40% active as supplied) (Magnesium Elektron Inc., Flemington, N.J.). The webs were dried and weighed to determine % superabsorbent polymer present in the composite.

The samples of Control 1 and Examples 1-4 were tested according to the above-described methods to determine wet and dry tensile strength. The weight and thickness of the samples were also determined. The results are reported in Table 1.

TABLE 1

| | | | | Dry | | Tensile | Tensile |
|---|---|---|---|---|---|---|---|
| | | | | Composite | Wet | Strength | Strength |
| | | | | Basis | Composite | (Dry) | (Wet) |
| | Basis | Thickness | | Weight | Thickness | g/25.4 | g/25.4 |
| Sample | Weight (g/m²) | (mm) | % SAP | (g/m²) | (mm) | mm | mm |
| Control 1 | 22 | 0.06 | 82 | 122 | 1.5 | 2820 | 480 |
| Example 1 | 30 | 2 | 83 | 176 | 2.3 | 2430 | 385 |
| Example 2 | 60 | 5 | 73 | 222 | 11 | 2480 | 410 |
| Example 3 | 60 | 5 | 90 | 600 | 13 | 2660 | 460 |
| Example 4 | 100 | 14 | 76 | 416 | 16 | 2870 | 406 |

### Examples 5-17

Superabsorbent composites were prepared according to Example 1 with the exception that the nonwoven webs had the basis weight and density set forth in Table 2 and the amount of superabsorbent polymer applied to the web was controlled to achieve a composite having the % superabsorbent polymer indicated in Table 2.

The samples of Controls 1 and 2 and Examples 5-17 were tested according to the above-described methods to determine the water absorbent capacity and 0.9% Saline absorbency under load (AUL). The results are reported in Table 2.

TABLE 2

| | | | Untreated Web | | SAP-Containing Composite | | |
|---|---|---|---|---|---|---|---|
| | | | | 0.9% Saline | | | 0.9% Saline |
| | | | | AUL | | | AUL |
| | | | Water | (g 0.9% | | Water | (g 0.9% |
| | Basis | | Absorbency | saline | | Absorbency | saline |
| | Weight | Density | (g water/g | solution/g | | (g water/g | solution/g |
| Sample | (g/m²) | (g/cm³) | composite) | composite) | % SAP | composite) | composite) |
| Control 1 | 22 | ND | 4 | 2 | 82 | 18 | 10 |
| Example 5 | 30 | 0.0227 | 6 | 4 | 83 | 24 | 12 |
| Example 6 | 30 | 0.0227 | 6 | 4 | 71 | 18 | 10 |
| Example 7 | 30 | 0.0227 | 6 | 4 | 57 | 12 | 14 |
| Example 8 | 30 | 0.0227 | 6 | 4 | 52 | 10 | 14 |
| Example 9 | 60 | 0.0024 | 10 | 5 | 87 | 31 | 12 |
| Example 10 | 60 | 0.0076 | 18 | 5 | 90 | 65 | 17 |
| Example 11 | 60 | 0.0076 | 18 | 5 | 79 | 46 | 15 |
| Example 12 | 60 | 0.0076 | 18 | 5 | 73 | 37 | 15 |
| Example 13 | 60 | 0.0076 | 18 | 5 | 62 | 31 | 13 |
| Example 14 | 60 | 0.0076 | 18 | 5 | 50 | 28 | 14 |
| Example 15 | 100 | 0.0083 | 20 | 7 | 76 | 34 | 18 |
| Example 16 | 100 | 0.0083 | 20 | 7 | 59 | 33 | 17 |
| Example 17 | 100 | 0.0083 | 20 | 7 | 51 | 31 | 21 |
| Control 2 | 300 | 0.046 | 30 | 15 | 50 | 22 | 12 |

ND = not determined

### Example 18

A polyester fiber highloft nonwoven web, 10 cm×10 cm×1.1 cm in dimension, weighing 0.86 g, and having a density of 0.0078 g/cm³ (Carpenter Company, Temple, Tex.) was saturated with PD-8081-H aqueous superabsorbent polymer (H.B. Fuller Company), and dried. The treated web had a superabsorbent polymer loading of 78.6%. The treated web was then contacted with seventy-five ml of 0.9% saline solution applied at a rate of 7 ml/second using a variable flow pump manufactured by Manostat, a Division of Barnant Co. (Barrington, Ill.).

The web absorbed 75 ml of 0.9% saline solution. No leakage or overflow was observed. The surface of the web was wet to the touch. Gelled superabsorbent polymer was visible at the surface.

### Example 19

A polyester fiber highloft nonwoven web, 10 cm×10 cm×1.5 cm in dimension, weighing 0.96 g, and having a density of 0.0064 g/cm³ (Carpenter Company) was obtained for use in Example 19. A first region of the web was saturated with PD-8081-H aqueous superabsorbent polymer such that the region containing superabsorbent polymer extended from a first major surface of the web into a portion of the depth of the web. The aqueous superabsorbent polymer was allowed to dry. The superabsorbent polymer loading on the web was 85.8%. A second region extending from the second major surface of the web opposite the first major surface and the first region into a portion of the depth of the web was free of superabsorbent polymer. The treated web was placed between two glass plates such that the region of the web that the second major surface, i.e., the region that was free of superabsorbent polymer, was in contact with the top glass plate. The top glass plate included a hole. Seventy-five ml of 0.9% saline solution was applied to the web through the hole at a rate of 7 ml/second using a variable flow pump manufactured by Manostat, a Division of Barnant Co. (Barrington, Ill.).

### Example 20

A polyester fiber highloft nonwoven web, 9.8 cm×9.5 cm×2.0 cm in dimension, weighing 1.66 g and having a density of 0.0089 g/cm³ (Carpenter Company) was obtained for use in was obtained for Example 20. A first region of the web was saturated with PD-8081-H aqueous superabsorbent polymer such that the region containing superabsorbent polymer extended from a first major surface of the web into a portion of the depth of the web. The aqueous superabsorbent polymer was allowed to dry. The superabsorbent polymer loading on the web was 65.6%. A second region extending from the second major surface of the web opposite the first major surface and the first region into a portion of the depth of the web was free of superabsorbent polymer. The treated web was placed between two glass plates such that the region of the web that the second major surface, i.e., the region that was free of superabsorbent polymer, was in contact with the top glass plate. The top glass plate included a hole. Seventy-five ml of 0.9% saline solution was applied to the web through the hole at a rate of 7 ml/second using a variable flow pump manufactured by Manostat, a Division of Barnant Co. (Barrington, Ill.).

The webs of Examples 19 and 20 absorbed the 75 ml of 0.9% saline solution. No leakage or overflow was observed. The second major surface of the webs exhibited an overall dry feeling except in the area where the saline solution was introduced into the web. No gelled superabsorbent polymer was visible on the second major surface of the webs.

Other embodiments are within the claims. Although the superabsorbent composite has been described with respect to disposable article cores, the superabsorbent composite is also useful in various other absorbent article applications including, e.g., wipes, towels, facial tissue, mops, and agricultural applications (e.g., to maintain moisture). The composite can also be combined with at least one other nonwoven web in a layered construction.

What is claimed is:

1. An absorbent article comprising:
a nonwoven web comprising
synthetic polymer fibers;
a first surface;
a second surface opposite said first surface;
a first region comprising superabsorbent polymer, said first region extending into said nonwoven web from said first surface toward said second surface, said superabsorbent polymer having been formed in situ in said first region; and
a second region essentially free of superabsorbent polymer, said second region extending into said nonwoven web from said second surface toward said first surface.

2. The absorbent article of claim 1, wherein said second region extends into the thickness of said nonwoven web a distance sufficient to prevent the wearer of said article from contacting said superabsorbent polymer of said first region after said superabsorbent polymer has been contacted with liquid.

3. The absorbent article of claim 1, wherein said first region is impregnated with said superabsorbent polymer.

4. The absorbent article of claim 1, wherein said polymer comprises polyester, polyolefin, polyamide, polyurethane, polyacrylonitrile, or a combination thereof.

5. The absorbent article of claim 1, wherein said second region exhibits a property comprising fluid acquisition, fluid dispersion or a combination thereof.

6. The absorbent article of claim 1, wherein said second region is available for contact with the wearer of said absorbent article.

7. The absorbent article of claim 1, wherein said first region is capable of storing fluid.

8. The absorbent article of claim 1, wherein said second region comprises a first plurality of fibers comprising a first polymer and a second plurality of fibers comprising a second polymer.

9. The absorbent article of claim 1, wherein said second region is soft to the touch.

10. The absorbent article of claim 1, wherein said first region comprises fibers comprising a first polymer and said second region comprises fibers comprising a second polymer.

11. The absorbent article of claim 1 further comprising cellulose fibers.

12. The absorbent article of claim 11, wherein said cellulose fibers are in contact with said first region.

13. The absorbent article of claim 1, wherein said absorbent article comprises
a top sheet; and
a back sheet;
said nonwoven web being disposed between said top sheet and said back sheet.

14. The absorbent article of claim 13, wherein said back sheet is positioned away from a wearer's skin.

15. The absorbent article of claim 13, wherein said back sheet is liquid impervious.

16. The absorbent article of claim 13, wherein said back sheet is moisture vapor permeable.

17. The absorbent article of claim 1, wherein said first region is substantially coextensive with said second region.

18. The absorbent article of claim 1, wherein said article is free of at least one of a fluid acquisition sheet and a fluid dispersion sheet.

19. A disposable diaper, feminine hygiene product, bandage, wound care product, surgical pad, adult incontinence pad, breast pad, bib or mattress pad comprising the absorbent article of claim 1.

20. An absorbent article comprising:
an absorbent core comprising a nonwoven web comprising
synthetic polymer fibers;
a first surface;
a second surface opposite said first surface;
a first region comprising superabsorbent polymer, said first region extending into said nonwoven web from said first surface toward said second surface, said superabsorbent polymer having been formed in situ in said first region; and
a second region essentially free of superabsorbent polymer, said second region extending into said nonwoven web from said second surface toward said first surface.

21. An absorbent core dimensioned for use in an absorbent article, said core comprising a nonwoven web comprising
synthetic polymer fibers,
a liquid storage region comprising a superabsorbent polymer, said liquid storage region extending into said nonwoven web from a first surface of said nonwoven web toward a second surface of said nonwoven web, said superabsorbent polymer having been formed in situ in said liquid storage region; and
liquid acquisition region free of superabsorbent polymer, said liquid acquisition region extending into said nonwoven web from a first surface of said nonwoven web toward said liquid storage region.

22. An article comprising a nonwoven web comprising
a first surface,
a second surface opposite said first surface,
a fluid storage region comprising superabsorbent polymer, said fluid storage region extending into said nonwoven web from said second surface of said web toward said first surface of said web, said superabsorbent polymer having been formed in situ in said first region; and
a fluid acquisition region essentially free of superabsorbent polymer, said fluid acquisition region extending into said nonwoven web from said first surface toward said second surface.

23. A method of making an absorbent article, said method comprising:
impregnating a first region of a nonwoven web with an aqueous composition comprising a superabsorbent polymer precursor and a crosslinking agent, said first region extending from a first surface of said web into the depth of the web such that the second surface of said web opposite said first surface of said web is free of superabsorbent polymer precursor; and
drying said composition.

24. The method of claim 23, wherein said drying comprises exposing said composition to microwave radiation.

25. The method of claim 23, wherein said drying comprises exposing said composition to heat.

26. A method of making an absorbent article, said method comprising:
impregnating a high loft nonwoven web with an aqueous composition comprising a superabsorbent polymer precursor and a crosslinking agent; and
drying said composition, said drying comprising exposing said composition to microwave radiation,
the dried absorbent article comprising from 10% by weight to about 90% by weight superabsorbent polymer.
